(19) United States
(12) Patent Application Publication
(10) Pub. No.: US 2022/0142205 A1
(43) Pub. Date: May 12, 2022

(54) FEED FOR ENHANCING IMMUNITY OF TILAPIA AND PREPARATION METHOD THEREOF

(71) Applicant: GUANGDONG OCEAN UNIVERSITY, Zhanjiang (CN)

(72) Inventors: Bei Wang, Zhanjiang (CN); Zhongliang Wang, Zhanjiang (CN); Yu Huang, Zhanjiang (CN); Shuanghu Cai, Zhanjiang (CN); Jichang Jian, Zhanjiang (CN)

(21) Appl. No.: 17/451,776

(22) Filed: Oct. 21, 2021

Related U.S. Application Data
(63) Continuation-in-part of application No. PCT/CN2021/088696, filed on Apr. 21, 2021.

(30) Foreign Application Priority Data
Nov. 11, 2020 (CN) 202011255097.2

(51) Int. Cl. A23K 50/80; A61K 38/54; A61K 31/7028; A23K 10/38; A23K 10/22; A23K 10/26; A23K 20/163; A23K 20/105; A23K 20/189; A23K 20/142; A23K 20/174; A23K 20/20; A23K 40/10

(52) U.S. Cl. CPC A23K 50/80; A61K 38/54; A61K 31/7028; A23K 10/38; A23K 10/22; A23K 10/26; A23K 40/10; A23K 20/105; A23K 20/189; A23K 20/142; A23K 20/174; A23K 20/30; A23K 20/163

(57) ABSTRACT

A feed for enhancing immunity of tilapia and preparation method thereof, wherein the feed includes 20-30 parts of rice chaff, 15-25 parts of earthworm powder, 10-15 parts of shrimp shell powder, 10-15 parts of sodium alginate, 5-10 parts of oyster shell powder, 5-10 parts of cyclohexanone, 10-15 parts of honeysuckle extract, 3-5 parts of Commelina communis extract, 3-6 parts of β-1,3-glucan, 2-3 parts of flavomycin growth promoter, 1-3 parts of complex enzyme preparation, 5-8 parts of selenium methionine, 6-10 parts of distiller's grains, 1-3 parts of complex vitamins, 1-3 parts of complex trace elements and 2-3 parts of food attractant, in parts by weight.

### CROSS REFERENCE TO RELATED APPLICATIONS

This application is a Continuation-in-Part of International Application No. PCT/CN2021/088696, filed Apr. 4, 2021, which claims priority to Chinese Patent Application No. 202011255097.2, entitled “feed for enhancing immunity of tilapia and preparation method thereof” filed with the China National Intellectual Property Administration on Nov. 11, 2020, are of which are incorporated herein by reference in their entireties.

### TECHNICAL FIELD

The present disclosure belongs to the technical field of feed, especially relating to a feed for enhancing immunity of tilapia and preparation method thereof.

### BACKGROUND ART

At present, aquaculture is an advantage industry in China with broad prospects for development. In recent years, the aquaculture has grown rapidly. However, the every occurrence of a major aquatic epidemic, such as the recent epidemics of early mortality syndrome, gill disease, enteritis and other bacterial and viral diseases with high infection, high pathogenicity and high mortality caused by bacteria and viruses, is a serious blow to China's aquaculture industry. It is estimated that the direct loss caused by the death of aquatic animals caused by the diseases is equivalent to about 60% of the increase in the total output value of aquaculture in China every year.

In the production of cultured fish, antibiotics are commonly used to prevent bacterial infections and promote the growth of animals. However, antibiotics kill harmful bacteria and also kill beneficial bacteria. The long-term use of antibiotics not only makes bacteria resistant, but also leaves drug residues and the impact on the quality of livestock products as a serious problem.

The existing feeds for aquatic animals have the defects of single nutrition and unreasonable nutrient collocation, which cannot effectively enhance immunity and cannot prevent and reduce the incidence of aquatic animals. In addition, the feed utilization rate is low and the feed stability is poor.

Through the above analysis, the existing problems and defects of the prior art are as follows:

(1) The existing feeds for aquatic animals have the defects of single nutrition and unreasonable nutrient collocation, which cannot effectively enhance immunity and cannot prevent and reduce the incidence of aquatic animals. In addition, the feed utilization rate is low and the feed stability is poor.

(2) The existing methods for preventing and controlling bacteria by using antibiotics lead to strong drug resistance of pathogenic bacteria and the food safety can't be guaranteed.

### SUMMARY OF THE INVENTION

According to the defects existing in the prior art, the present disclosure provides a feed for enhancing immunity of tilapia and preparation method thereof.

The present disclosure provides a feed for enhancing immunity of tilapia, wherein the feed comprises 20-30 parts of rice chaff, 15-25 parts of earthworm powder, 10-15 parts of shrimp shell powder, 10-15 parts of sodium alginate, 5-10 parts of oyster shell powder, 5-10 parts of cyclohexanone, 10-15 parts of honeysuckle extract, 3-5 parts of Commelina communis extract, 3-6 parts of β-1,3-glucan, 2-3 parts of flavomycin growth promoter, 1-3 parts of complex enzyme preparation, 5-8 parts of selenium methionine, 6-10 parts of distiller's grains, 1-3 parts of complex vitamins, 1-3 parts of complex trace elements and 2-3 parts of food attractant, in parts by weight.

In some embodiments, the complex enzyme preparation is a mixture of amylase, protease and cellulase, and the weight ratio of amylase, protease and cellulase is 3:2:1.

In some embodiments, the complex vitamins comprise two or more of vitamin C, vitamin E, vitamin K, vitamin A, vitamin D3, nicotinic acid, folic acid, inositol, and calcium pantothenate.

In some embodiments, the complex trace elements include iron, copper, manganese and iodine.

In some embodiments, the food attractant comprises allicin, seaweed, straw mushroom powder, dried silkworm pupa, clam meat, zeolite powder and fish soluble.

In some embodiments, the weight ratio of allicin, seaweed, straw mushroom powder, dried silkworm pupa, clam meat, zeolite powder and fish soluble is 2:2:1:1:1:1:2.

Another object of the present disclosure is to provide a method for preparing the feed for enhancing immunity of tilapia, wherein the method includes the steps of:

S1: weighing rice chaff, earthworm powder, shrimp shell powder, sodium alginate, oyster shell powder, cyclohexanone, honeysuckle, Commelina communis, flavomycin growth promoter, complex enzyme preparation, selenium methionine, distiller's grains, complex vitamins and complex trace elements in proportion; preparing honeysuckle extract, Commelina communis extract and food attractant respectively;

S2: extracting β-1,3-glucan; mixing weighed rice chaff, earthworm powder, shrimp shell powder, and oyster shell powder evenly after removing impurities, crushing and passing through a 120-mesh sieve to obtain a mixed powder A;

S3: grinding sodium alginate, cyclohexanone and selenium methionine to obtain a mixture B; adding clear water, mixed powder A and mixture B to distiller's grains to obtain a mixture C after stirring evenly;

S4: adding flavomycin growth promoter, complex enzyme preparation, complex vitamins, complex trace elements to the mixture C to obtain feed for enhancing the immunity of tilapia after stirring evenly, granulating and drying.

In some embodiments, after grinding sodium alginate, cyclohexanone and selenium methionine, the method for preparing the feed for enhancing immunity of tilapia further includes the step of mixing with honeysuckle extract, Commelina communis extract, food attractant and β-1,3-glucan to obtain a mixture B.

In some embodiments, a method for preparing the honeysuckle extract in S1 comprises the steps of:

(1) cleaning and drying honeysuckle; cutting the dried honeysuckle into 1-3 cm segments, adding 15 times of water, and decocting the honeysuckle to obtain a primary decoction;

(2) vacuuming the primary decoction, heating to 60-80° C. to make the primary decoction boil, and keeping temperature for 1-3 h, and then filtering to obtain a primary filtrate and a primary filter residue;

(3) adding extracting solvent to the primary filter residue for immersion and performing reflux extraction to obtain an extract; after filtering, mixing the filtered extract and the primary filtrate, concentrating the resulting mixture under reduced pressure, and drying the resulting concentrated solution to obtain the honeysuckle extract.

In some embodiments, the ratio of the primary filter residue and extracting solvent in the step 3) is 1:15.

In some embodiments, the parameters of reflux extraction in the step 3) are: temperature of the reflux extraction is 55° C.-95° C., extraction times are 2-3 times, and extraction time is 0.5 h-2 h.

In some embodiments, the extracting solvent in the step 3) is an ethanol aqueous solution with a volume concentration of 75%.

In some embodiments, a method for preparing the Commelina communis extract in S1 comprises the steps of:

firstly, steaming the Commelina communis at high temperature, collecting a steamed solution and a steamed Commelina communis;

secondly, putting the steamed Commelina communis into a beating machine to obtain a Commelina communis slurry;

then, heating and concentrating the steamed solution to obtain a concentrated solution;

finally, mixing the Commelina communis slurry and the concentrated solution to obtain a Commelina communis extract.

In some embodiments, the temperature of steaming is 100° C.

In some embodiments, a method for preparing the food attractant in S1 comprises the steps of:

1) weighing allicin, seaweed, straw mushroom powder, dried silkworm pupa, clam meat, zeolite powder and fish soluble; grinding allicin, seaweed, straw mushroom powder and zeolite powder, passing through a 140-mesh sieve to obtain a mixed powder;

2) injecting the fish soluble into a 500 L stainless steel vessel and steaming at high temperature for 30 min-60 min, natural cooling to 30° C.;

3) adding dried silkworm pupa and clam meat to the cooled fish soluble, mixing fully and adding trypsin and papain, mixing fully and fermenting at 37° C. for 3 h-5 h;

4) heating the fermentation product to boil for concentration until the water content is 63%, and cooling a concentrated fermentation product to 37° C.; adding mixed powder to a cooled mixture and drying in a vacuum drying oven to obtain the food attractant.

In some embodiments, a method for extracting the β-1,3-glucan in the S2 comprises the steps of:

(2.1) washing, drying and crushing the mushroom, passing through a 120-mesh sieve to obtain a mushroom powder; dissolving the mushroom powder in 4-6 times volume of 100 mM phosphate buffer with pH 6.0-6.5, and extracting at 6° C. for 6 h;

(2.2) high-speed centrifuging to remove precipitate, saving supernatant to obtain a crude extract of β-1,3-glucan.

(2.3) adding a certain amount of NaOH into the crude extract of β-1,3-glucan, raising the temperature to 60° C.-75° C. to react for 2 h-3 h, collecting precipitate by filtration;

(2.4) adding acid solution to the precipitate, adjusting the pH to 5.5, raising the temperature to 65° C. to react for 1 h-2 h, collecting precipitate by filtration;

(2.5) washing the precipitate, extracting with acetone and freeze-drying to obtain the β-1,3-glucan.

In some embodiments, the amount of the NaOH in the step (2.3) added adjusts the pH of the crude extract to 6.0-6.5.

Combining all the above technical solutions, the advantages and positive effects of the present disclosure are:

The feed of the present disclosure takes rice chaff, earthworm powder, shrimp shell powder, sodium alginate, oyster shell powder, cyclohexanone, honeysuckle, Commelina communis, flavomycin growth promoter, complex enzyme preparation, selenium methionine, distiller's grains, complex vitamins and complex trace elements as raw materials, the raw material formula has rich nutrition and good palatability, which provides protein, amino acid, dietary fiber and mineral matter for the growth and development of aquatic animals, and is easy to digest and absorb by aquatic animals. The feed of the present disclosure not only promotes the growth and development of aquatic animals, but also effectively improves the physique of aquatic animals Therefore, the feed of the present disclosure enhances the immunity of aquatic animals, has good disease resistance and low morbidity, thus effectively reducing the risk of aquatic animal breeding.

The feed of the present disclosure has rich nutritional structure and high nutritional value, may effectively regulate the metabolism of aquatic animals, improve the cell activity of aquatic animals, enhance immunity and anti-stress ability of aquatic animals, improve the physical fitness of aquatic animals, effectively promote the growth and development of aquatic animals, thus improving the survival rate yield and meat quality of aquatic animals. In addition, the product of the present disclosure has stable performance, may effectively inhibit pathogenic bacteria, reduce the incidence of aquatic animals, and promote the efficient absorption of nutrients by aquatic animals.

The feed of the present disclosure may control the growth of various bacterium and viruses, remove the toxins in the blood, and may also eliminate the bacterial virions invaded from the outside of the body, thus realizing the function of clearing heat and resolving toxin. It may also kill a variety of infectious fungi, bacterium, and inhibit and eliminate the growth and reproduction of pathogens, improve immunity of aquatic animals and promote the recovery of aquatic animals. Especially in the early stage of the disease, the feed of the present disclosure may significantly alleviate symptoms, control the deterioration of the disease, and make aquatic animals develop immunity.

The food attractant added in the feed of the present disclosure may obviously promote the ingestion of aquatic animals, promote the growth of aquatic animals, reduce residual feed, reduce or avoid the use of fish meal, thereby reducing aquaculture costs. The nutrients of feed are easy to absorb by aquatic animals, and have a protective effect on the intestines of aquatic animals.

### BRIEF DESCRIPTION OF THE DRAWINGS

In order to illustrate the technical solutions of the embodiments of the present disclosure more clearly. The present disclosure will briefly introduce the drawings of the embodiments. Obviously, the drawings described below are only some embodiments of the present disclosure. For those of ordinary skills in the art, other drawings can be obtained from these drawings without creative work.

FIG. 1 is a flow chart of the method for preparing the feed for enhancing immunity of tilapia provided by the embodiments of the present disclosure.

FIG. 2 is a flow chart of the method for preparing the honeysuckle extract provided by the embodiments of the present disclosure.

FIG. 3 is a flow chart of the method for preparing the Commelina communis extract provided by the embodiments of the present disclosure.

FIG. 4 is a flow chart of the method for preparing the food attractant provided by the embodiments of the present disclosure.

FIG. 5 is a flow chart of the method for extracting the β-1,3-glucan provided by the embodiments of the present disclosure.

FIG. 6A shows the effect of different formulas of feed on serum biochemical index of tilapia on day 7.

FIG. 6B shows the effect of different formulas of feed on serum biochemical index of tilapia on day 14.

FIG. 6C shows the effect of different formulas of feed on serum biochemical index of tilapia on day 21.

FIG. 6D shows the effect of different formulas of feed on serum biochemical index of tilapia on day 28.

FIG. 6E shows the effect of different formulas of feed on serum biochemical index of tilapia on day 35.

FIG. 7A shows the effect of different formulas of compound Chinese herbal medicine on serum immune index of tilapia on day 7.

FIG. 7B shows the effect of different formulas of compound Chinese herbal medicine on serum immune index of tilapia on day 14.

FIG. 7C shows the effect of different formulas of compound Chinese herbal medicine on serum immune index of tilapia on day 21.

FIG. 7D shows the effect of different formulas of compound Chinese herbal medicine on serum immune index of tilapia on day 28.

FIG. 7E shows the effect of different formulas of compound Chinese herbal medicine on serum immune index of tilapia on day 35.

FIG. 8A shows the effect of different formulas of compound Chinese herbal medicine on the intestinal flora index of tilapia on day 7.

FIG. 8B shows the effect of different formulas of compound Chinese herbal medicine on the intestinal flora index of tilapia on day 14.

FIG. 8C shows the effect of different formulas of compound Chinese herbal medicine on the intestinal flora index of tilapia on day 21.

FIG. 8D shows the effect of different formulas of compound Chinese herbal medicine on the intestinal flora index of tilapia on day 28.

FIG. 8E shows the effect of different formulas of compound Chinese herbal medicine on the intestinal flora index of tilapia on day 35.

FIG. 9 shows the effect of different formulas of compound Chinese herbal medicine on the immune protection of tilapia against Streptococcus agalactiae.

### DETAILED DESCRIPTION OF THE EMBODIMENTS

In order to make the object, technical solution and advantages of the present disclosure more clear, the present disclosure will be further illustrated in detail with the embodiments. It should be understood that the specific embodiments described herein are only intended to illustrate the disclosure, but should not be regarded as limiting the present disclosure.

In view of the problems in the prior art, the present disclosure provides a feed for enhancing the immunity of tilapia and preparation method thereof, and the present disclosure is described in detail in combination with the attached drawings.

The feed for enhancing the immunity of tilapia provided by the embodiments of the present disclosure comprises 20-30 parts of rice chaff, 15-25 parts of earthworm powder, 10-15 parts of shrimp shell powder, 10-15 parts of sodium alginate, 5-10 parts of oyster shell powder, 5-10 parts of cyclohexanone, 10-15 parts of honeysuckle extract, 3-5 parts of Commelina communis extract, 3-6 parts of β-1,3-glucan, 2-3 parts of flavomycin growth promoter, 1-3 parts of complex enzyme preparation, 5-8 parts of selenium methionine, 6-10 parts of distiller's grains, 1-3 parts of complex vitamins, 1-3 parts of complex trace elements and 2-3 parts of food attractant, in parts by weight.

The complex enzyme preparation provided by the embodiments of the present disclosure is a mixture of amylase, protease, and cellulase, and the weight ratio of amylase, protease and cellulase is 3:2:1.

The complex vitamins provided in the embodiments of the present disclosure are two or more of vitamin C, vitamin E, vitamin K, vitamin A, vitamin D3, nicotinic acid, folic acid, inositol, and calcium pantothenate.

The complex trace elements provided by the embodiments of the present disclosure include iron, copper, manganese and iodine.

The food attractant provided by the embodiments of the present disclosure comprises allicin, seaweed, straw mushroom powder, dried silkworm pupa, clam meat, zeolite powder and fish soluble. The weight ratio of allicin, seaweed, straw mushroom powder, dried silkworm pupa, clam meat, zeolite powder and fish soluble is 2:2:1:1:1:1:2.

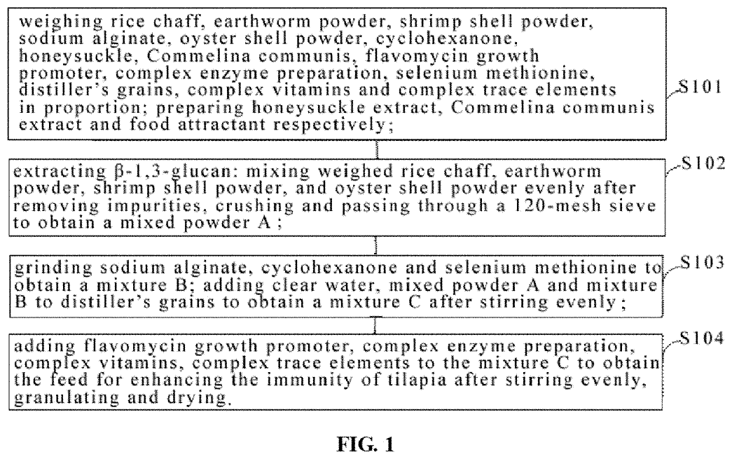

As shown in FIG. 1, the method for preparing the feed for enhancing immunity of tilapia provided by the embodiments of the present disclosure includes the steps of:

S101, weighing rice chaff, earthworm powder, shrimp shell powder, sodium alginate, oyster shell powder, cyclohexanone, honeysuckle, Commelina communis, flavomycin growth promoter, complex enzyme preparation, selenium methionine, distiller's grains, complex vitamins and complex trace elements in proportion; preparing honeysuckle extract, Commelina communis extract and food attractant respectively;

S102, extracting β-1,3-glucan: mixing weighed rice chaff, earthworm powder, shrimp shell powder, and oyster shell powder evenly after removing impurities, crushing and passing through a 120-mesh sieve to obtain a mixed powder A;

S103, grinding sodium alginate, cyclohexanone and selenium methionine to obtain a mixture B; adding clear water, mixed powder A and mixture B to distiller's grains to obtain a mixture C after stirring evenly;

S104, adding flavomycin growth promoter, complex enzyme preparation, complex vitamins, complex trace elements to the mixture C to obtain the feed for enhancing the immunity of tilapia after stirring evenly, granulating and drying.

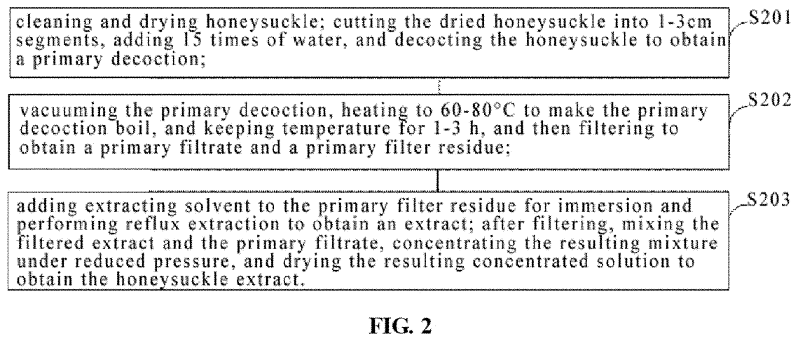

As shown in FIG. 2, the method for preparing the honeysuckle extract in step 101 provided by the embodiments of the present disclosure includes the steps of:

S201, cleaning and drying honeysuckle; cutting the dried honeysuckle into 1-3 cm segments, adding 15 times of water, and decocting the honeysuckle to obtain a primary decoction;

S202, vacuuming the primary decoction, heating to 60-80° C. to make the primary decoction boil, and keeping temperature for 1-3 h, and then filtering to obtain a primary filtrate and a primary filter residue;

S203, adding extracting solvent to the primary filter residue for immersion and performing reflux extraction to obtain an extract; after filtering, mixing the filtered extract and the primary filtrate, concentrating the resulting mixture under reduced pressure, and drying the resulting concentrated solution to obtain the honeysuckle extract.

The ratio of the primary filter residue and extracting solvent in the step S203 provided by the embodiments of the present disclosure is 1:15.

The parameters of reflux extraction in the step S203 provided by the embodiments of the present disclosure are: temperature of the reflux extraction is 55° C.-95° C., extraction times are 2-3 times, and extraction time is 0.5 h-2 h.

The extracting solvent in the step 3) is an ethanol aqueous solution with a volume concentration of 75%.

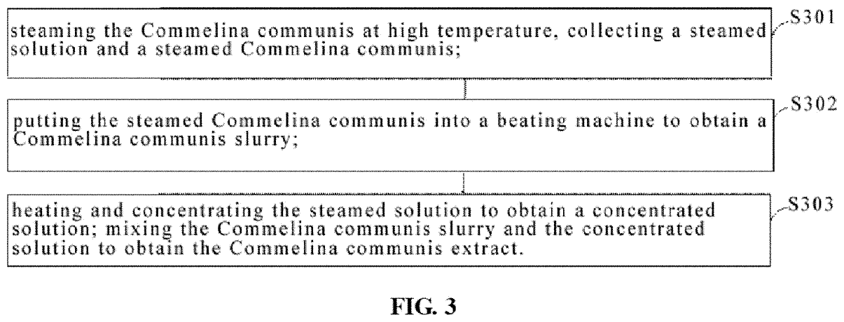

As shown in FIG. 3, a method for preparing the Commelina communis extract in step S101 provided by the embodiments of the present disclosure includes the steps of:

S301, steaming the Commelina communis at high temperature, collecting a steamed solution and a steamed Commelina communis; wherein the temperature of steaming is 100° C.;

S302, putting the steamed Commelina communis into a beating machine to obtain a Commelina communis slurry;

S303, heating and concentrating the steamed solution to obtain a concentrated solution; mixing the Commelina communis slurry and the concentrated solution to obtain the Commelina communis extract.

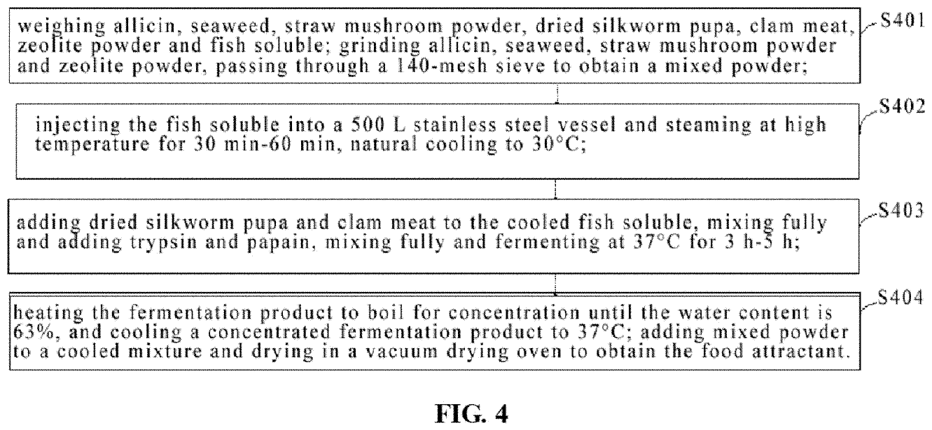

As shown in FIG. 4, the method for preparing the food attractant in step S101 provided by the embodiments of the present disclosure includes the steps of:

S401, weighing allicin, seaweed, straw mushroom powder, dried silkworm pupa, clam meat, zeolite powder and fish soluble; grinding allicin, seaweed, straw mushroom powder and zeolite powder, passing through a 140-mesh sieve to obtain a mixed powder;

S402, injecting the fish soluble into a 500 L stainless steel vessel and steaming at high temperature for 30 min-60 min, natural cooling to 30° C.;

S403, adding dried silkworm pupa and clam meat to the cooled fish soluble, mixing fully and adding trypsin and papain, mixing fully and fermenting at 37° C. for 3 h-5 h;

S404, heating the fermentation product to boil for concentration until the water content is 63%, and cooling a concentrated fermentation product to 37° C.; adding mixed powder to a cooled mixture and drying in a vacuum drying oven to obtain the food attractant.

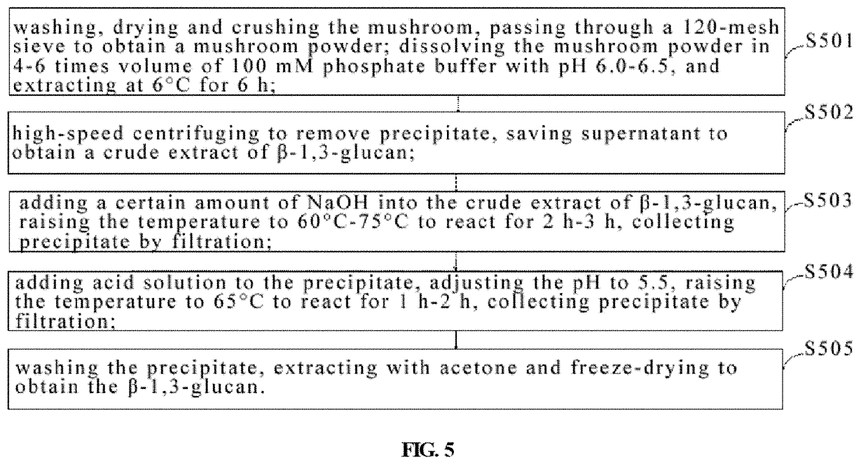

As shown in FIG. 5, the method for extracting the β-1,3-glucan in the step S102 includes the steps of:

S501, washing, drying and crushing the mushroom, passing through a 120-mesh sieve to obtain a mushroom powder; dissolving the mushroom powder in 4-6 times volume of 100 mM phosphate buffer with pH 6.0-6.5, and extracting at 6° C. for 6 h;

S502, high-speed centrifuging to remove precipitate, saving supernatant to obtain a crude extract of β-1,3-glucan;

S503, adding a certain amount of NaOH into the crude extract of β-1,3-glucan, raising the temperature to 60° C.-75° C. to react for 2 h-3 h, collecting precipitate by filtration;

S504, adding acid solution to the precipitate, adjusting the pH to 5.5, raising the temperature to 65° C. to react for 1 h-2 h, collecting precipitate by filtration;

S505, washing the precipitate, extracting with acetone and freeze-drying to obtain the β-1,3-glucan.

The feed for enhancing immunity of tilapia of the present disclosure will be further described in detail below in combination with examples, but should not be regarded as limiting the scope of the present disclosure.

### Example 1

1. The feed for enhancing the immunity of tilapia provided by the embodiments of the present disclosure was prepared into five different formulas of tilapia feeds A, B, C, D and E in a certain proportion, and the formulas of each group of feeds were as follows:

The group A of tilapia feed included rice chaff 30 kg, earthworm powder 25 kg, shrimp shell powder 15 kg, sodium alginate 15 kg, oyster shell powder 10 kg, cyclohexanone 10 kg, honeysuckle extract 10 kg, Commelina communis extract 3 kg, β-1,3-glucan 3 kg, flavomycin growth promoter 2 kg, complex enzyme preparation 1 kg, selenium methionine 5 kg, distiller's grains 10 kg, complex vitamins 1 kg, complex trace elements 1 kg and food attractant 2 kg.

The group B of tilapia feed included rice chaff 25 kg, earthworm powder 18 kg, shrimp shell powder 13 kg, sodium alginate 12 kg, oyster shell powder 8 kg, cyclohexanone 5 kg, honeysuckle extract 15 kg, Commelina communis extract 5 kg, β-1,3-glucan 6 kg, flavomycin growth promoter 3 kg, complex enzyme preparation 3 kg, selenium methionine 7 kg, distiller's grains 7 kg, complex vitamins 2 kg, complex trace elements 2 kg and food attractant 3 kg.

The group C of tilapia feed included rice chaff 25 kg, earthworm powder 22 kg, shrimp shell powder 10 kg, sodium alginate 10 kg, oyster shell powder 8 kg, cyclohexanone 8 kg, honeysuckle extract 12 kg, Commelina communis extract 4 kg, β-1,3-glucan 5 kg, flavomycin growth promoter 2 kg, complex enzyme preparation 2 kg, selenium methionine 6 kg, distiller's grains 9 kg, complex vitamins 2 kg, complex trace elements 3 kg and food attractant 3 kg.

The group D of tilapia feed included rice chaff 20 kg, earthworm powder 15 kg, shrimp shell powder 10 kg, sodium alginate 10 kg, oyster shell powder 5 kg, cyclohexanone 5 kg, honeysuckle extract 10 kg, Commelina communis extract 3 kg, β-1,3-glucan 3 kg, flavomycin growth promoter 2 kg, complex enzyme preparation 1 kg, selenium methionine 5 kg, distiller's grains 6 kg, complex vitamins 1 kg, complex trace elements 1 kg and food attractant 2 kg.

The group E of tilapia feed included rice chaff 20 kg, earthworm powder 15 kg, shrimp shell powder 10 kg, sodium alginate 10 kg, oyster shell powder 5 kg, cyclohexanone 5 kg, honeysuckle extract 15 kg, Commelina communis extract 5 kg, β-1,3-glucan 6 kg, flavomycin growth promoter 2 kg, complex enzyme preparation 3 kg, selenium methionine 8 kg, distiller's grains 10 kg, complex vitamins 3 kg, complex trace elements 3 kg and food attractant 3 kg.

At the same time, a control group of tilapia immunity feed was set up. The specific formula was as follows: 20 kg grass powder, 3 kg fish meal, 20 kg soybean cake, 20 kg corn gluten meal, 20 kg rice bran, 10 kg wheat bran, 5.5 kg cornmeal, 1 kg bone powder, 0.5 kg salt.

Herein, the complex enzyme preparation was a mixture of amylase, protease and cellulase, and the weight ratio of amylase, protease and cellulase is 3:2:1. The complex vitamins were vitamin C, vitamin E, vitamin K, vitamin A, vitamin D3, nicotinic acid, folic acid, inositol, and calcium pantothenate, and the weight ratio was 1:0.5:0.5:0.5:1:1:1:2:2.

The complex trace elements provided by the embodiments of the present disclosure included iron, copper, manganese and iodine.

The food attractant provided by the embodiments of the present disclosure included allicin, seaweed, straw mushroom powder, dried silkworm pupa, clam meat, zeolite powder and fish soluble. The weight ratio of allicin, seaweed, straw mushroom powder, dried silkworm pupa, clam meat, zeolite powder and fish soluble was 2:2:1:1:1:1:2.

2. The method for preparing the feed for enhancing immunity of tilapia provided by the embodiments of the present disclosure, as shown in FIG. 1, included the following steps:

S101, weighing rice chaff, earthworm powder, shrimp shell powder, sodium alginate, oyster shell powder, cyclohexanone, honeysuckle, Commelina communis, flavomycin growth promoter, complex enzyme preparation, selenium methionine, distiller's grains, complex vitamins and complex trace elements in proportion; preparing honeysuckle extract, Commelina communis extract and food attractant respectively;

S102, extracting β-1,3-glucan; mixing weighed rice chaff, earthworm powder, shrimp shell powder, and oyster shell powder evenly after removing impurities, crushing and passing through a 120-mesh sieve to obtain a mixed powder A;

S103, grinding sodium alginate, cyclohexanone and selenium methionine, mixing with honeysuckle extract, Commelina communis extract, food attractant and β-1,3-glucan to obtain a mixture B; adding clear water, mixed powder A and mixture B to distiller's grains to obtain a mixture C after stirring evenly;

S104, adding flavomycin growth promoter, complex enzyme preparation, complex vitamins, complex trace elements to the mixture C to obtain the feed for enhancing the immunity of tilapia after stirring evenly, granulating and drying.

The method for preparing the honeysuckle extract in step S101 provided by the embodiments of the present disclosure, as shown in FIG. 2, included the following steps:

S201, cleaning and drying honeysuckle; cutting the dried honeysuckle into 1-3 cm segments, adding 15 times of water, and decocting the honeysuckle to obtain a primary decoction;

S202, vacuuming the primary decoction, heating to 60-80° C. to make the primary decoction boil, and keeping temperature for 1-3 h, and then filtering to obtain a primary filtrate and a primary filter residue;

S203, adding extracting solvent to the primary filter residue for immersion and performing reflux extraction to obtain an extract; after filtering, mixing the filtered extract and the primary filtrate, concentrating the resulting mixture under reduced pressure, and drying the resulting concentrated solution to obtain the honeysuckle extract; the extracting solvent was an ethanol aqueous solution with a volume concentration of 75%.

The ratio of the primary filter residue and extracting solvent in the step S201 provided by the embodiments of the present disclosure was 1:15.

The parameters of reflux extraction in the step S203 provided by the embodiments of the present disclosure were: temperature of the reflux extraction was 55° C.-95° C., extraction times are 2-3 times, and extraction time was 0.5 h-2 h.

The method for preparing the Commelina communis extract in step S101 provided by the embodiments of the present disclosure, as shown in FIG. 3, included the following steps:

S301, steaming the Commelina communis at 100° C., collecting a steamed solution and a steamed Commelina communis;

S302, putting the steamed Commelina communis into a beating machine to obtain a Commelina communis slurry;

S303, heating and concentrating the steamed solution to obtain a concentrated solution; mixing the Commelina communis slurry and the concentrated solution to obtain the Commelina communis extract.

The method for preparing the food attractant in step S101 provided by the embodiments of the present disclosure, as shown in FIG. 4, included the following steps:

S401, weighing allicin, seaweed, straw mushroom powder, dried silkworm pupa, clam meat, zeolite powder and fish soluble; grinding allicin, seaweed, straw mushroom powder and zeolite powder, passing through a 140-mesh sieve to obtain a mixed powder;

S402, injecting the fish soluble into a 500 L stainless steel vessel and steaming at high temperature for 30 min-60 min, natural cooling to 30° C.;

S403, adding dried silkworm pupa and clam meat to the cooled fish soluble, mixing fully and adding trypsin and papain to make the final concentration of trypsin and papain in the mixture be 0.1%, mixing fully and fermenting at 37° C. for 3 h-5 h;

S404, heating the fermentation product to boil for concentration until the water content was 63%, and cooling a concentrated fermentation product to 37° C.; adding mixed powder to a cooled mixture and drying in a vacuum drying oven to obtain the food attractant.

The method for extracting the β-1,3-glucan in step S102 provided by the embodiments of the present disclosure, as shown in FIG. 5, included the following steps:

S501, washing, drying and crushing the mushroom, passing through a 120-mesh sieve to obtain a mushroom powder; dissolving the mushroom powder in 4-6 times volume of 100 mM phosphate buffer with pH 6.0-6.5, and extracting at 6° C. for 6 h;

S502, high-speed centrifuging to remove precipitate, saving supernatant to obtain a crude extract of β-1,3-glucan;

S503, adding a certain amount of NaOH into the crude extract of β-1,3-glucan to make the pH of the crude extract of β-1,3-glucan 6.0-6.5, raising the temperature to 60° C.-75° C. to react for 2 h-3 h, collecting precipitate by filtration;

S504, adding acid solution to the precipitate, adjusting the pH to 5.5, raising the temperature to 65° C. to react for 1 h-2 h, collecting precipitate by filtration;

S505, washing the precipitate, extracting with 2 times the volume of acetone and freeze-drying to obtain β-1,3-glucan.

### Example 2

The five different formulas of tilapia feed prepared in Example 1 were fed to tilapia. The feeding method was as follows: daily feed was fed at 09:00 and 17:00, and the daily feeding amount was 4% of the fish's body weight. The feeding amount was adjusted according to the growth status of the fish every week, and the feeding was stopped 24 hours before weighing the final body weight. During the whole process of the test, aeration was continuously performed to increase the oxygen content, and the water was changed once a day, the quantity of exchanged water was ⅔, and the temperature of the water was (28.5±1.5°) C. The optimal feed formula was screened by determining the weight gain rate, muscle composition, serum biochemical index, serum immune index, changes in intestinal flora index and the immune protection of tilapia against Streptococcus agalactiae; the specific test data were as follows:

1. The Effect of Different Formula Feeds on the Weight Gain Rate of Tilapia.

Test method: the weight gain rate (WGR) was calculated according to Formula I:

WGR=(final weight−initial weight)/initial weight (Formula I)

All data were analyzed by variance analysis using statistical methods.

The measurement results of weight gain rate show that the weight gain rate of the test groups are significantly higher than that of the control group after 5 weeks of feeding with different formula diets, and the weight gain rates of the test groups A, B, C, D and E increase by 42.53%, 49.28%, 44.72%, 38.19% and 41.08% respectively compared with the control group, with no significant difference between the test groups (P>0.05) (Table 1). The results show that different feeds A, B, C, D and E can significantly promote the growth of tilapia, but there is no significant difference in the weight gain rate of tilapia between the test groups.

TABLE 1
Effect of different formula feeds on the weight gain rate of tilapia

| | Control group | A | B | C | D | E |
|---|---|---|---|---|---|---|
| Weight gain rate | 257.87% | 367.54% | 384.95% | 373.19% | 356.35% | 363.8% |

2. Effects of Different Formula Feeds on Muscle Composition of Tilapia

Test method: the muscle composition of tilapia was determined according to China national standard GB/T 18654.10-2008 for the determination of muscle nutrient composition.

3 tilapia were randomly selected from each group for muscle composition analysis after feeding tilapia with different formulas for 5 weeks. The results show that there are significant differences in crude fat content and flavor nucleotides content between the test groups and the control group (P<0.05), but there are no significant differences in water, protamine and crude ash content between the test groups and the control group (P>0.05). The crude fat content in tilapia of the test groups A, B, C, D and E decrease by 30.68%, 32.87%, 34.52%, 28.21% and 32.05% respectively compared with the control group. There is significant difference between the test groups and the control group (P<0.05), but there is no significant difference between the test groups (P>0.05). There are significant differences in flavor nucleotides of tilapia in groups A, B, C and E compared with the control group (P<0.05). The flavor nucleotides of the test groups A, B, C and E are increased by 13.62%, 20.99%, 12.99% and 13.42% respectively compared with the control group. However, there is no significant difference between test group D and control group. This indicates that formula A, B, C and E can improve the meat quality of tilapia to a certain extent (Table 2).

TABLE 2
Effects of different culture feeds on muscle quality of tilapia

| | | | | | Inosinic | Flavor |
|---|---|---|---|---|---|---|
| | | | | | acid content | nucleotide |
| Groups | Water | Crude protein | Crude fat | Crude ash | (mg/g) | (mg/g) |
| Control | 78.22 ± 0.14 | 82.74 ± 0.27 | 3.65 ± 0.13^{a} | 6.15 ± 0.12 | 4.35 ± 0.15 | 4.62 ± 0.16^{a} |
| group | | | | | | |
| A | 74.16 ± 0.47 | 83.13 ± 0.27 | 2.53 ± 0.15^{b} | 6.07 ± 0.17 | 4.32 ± 0.17 | 5.25 ± 0.13^{b} |
| B | 75.23 ± 0.21 | 84.12 ± 0.44 | 2.45 ± 0.16^{b} | 6.13 ± 0.18 | 4.13 ± 0.18 | 5.59 ± 0.18^{c} |
| C | 73.98 ± 0.27 | 83.27 ± 0.34 | 2.39 ± 0.18^{b} | 5.99 ± 0.14 | 4.33 ± 0.21 | 5.22 ± 0.21^{b} |
| D | 75.81 ± 0.32 | 82.62 ± 0.19 | 2.62 ± 0.22^{b} | 6.17 ± 0.23 | 4.19 ± 0.13 | 4.91 ± 0.11^{a} |
| E | 76.63 ± 0.19 | 84.83 ± 0.25 | 2.48 ± 0.15^{b} | 5.95 ± 0.11 | 4.27 ± 0.20 | 5.24 ± 0.13^{b} |

3. Effects of Different Formula Feeds on Serum Biochemical Index of Tilapia

Test methods: blood samples were collected from the tail vein of tilapia. Glutamic-pyruvic transaminase (GPT), glutamic-oxaloacetate transaminase (GOT), triglyceride and total cholesterol were determined by Hitachi 7600-110 automatic biochemical analyzer. The test data were analyzed by SPSS 13.0 software, and the results were expressed as mean±standard deviation. The significance of the differences in each group was analyzed by using t test, and the difference was significant in P<0.05.

3 tilapia were randomly selected from each group for collecting blood samples from the tail vein. The blood was centrifuged at 4000 r/min for 10 min after standing at 4° C. overnight. The supernatant was collected and stored at −80° C. for later use. Glutamic-pyruvic transaminase (GPT), glutamic-oxaloacetate transaminase (GOT), triglyceride and total cholesterol were determined.

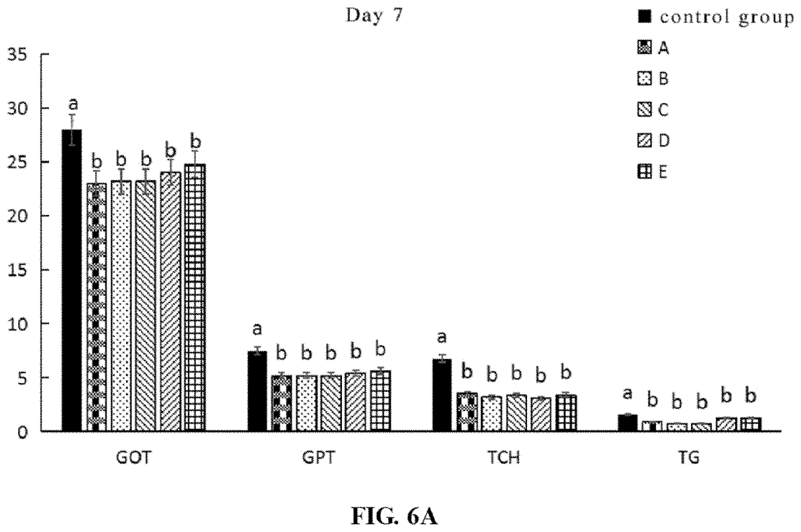

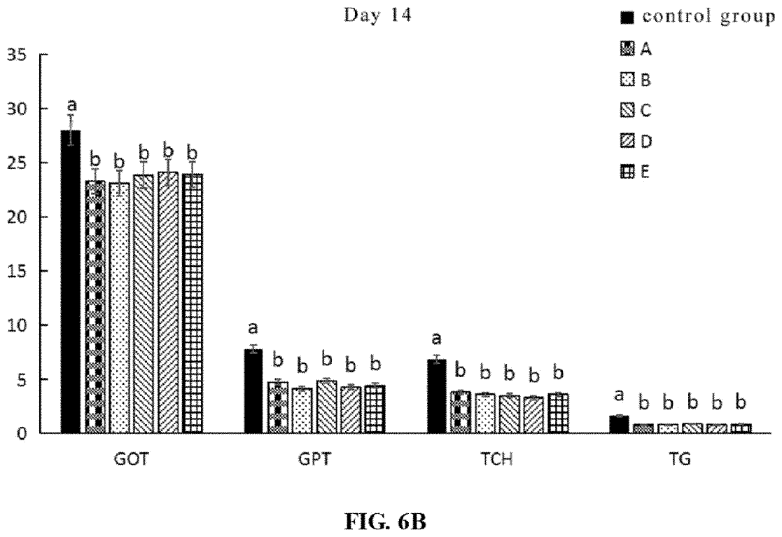

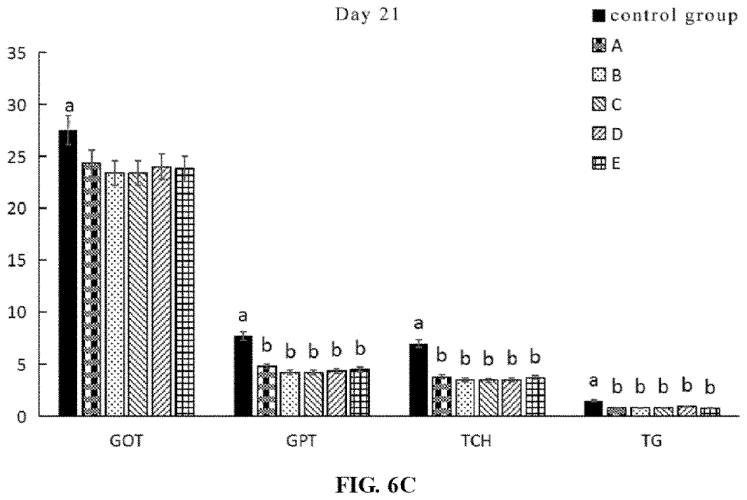

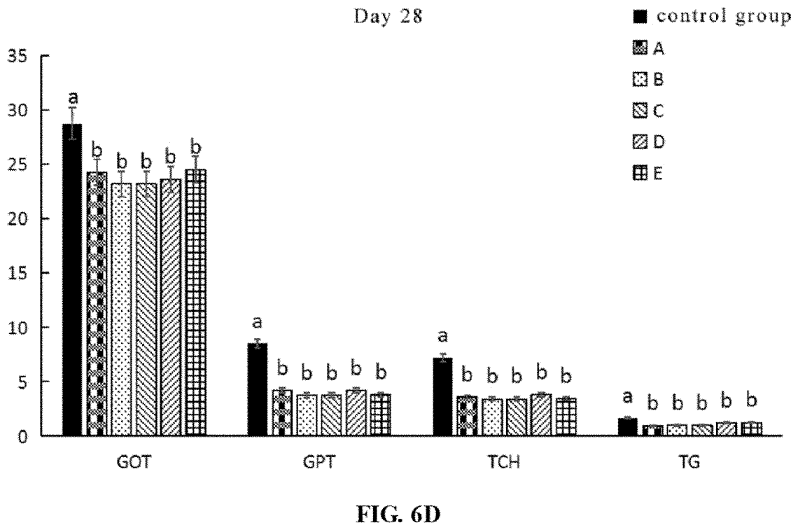

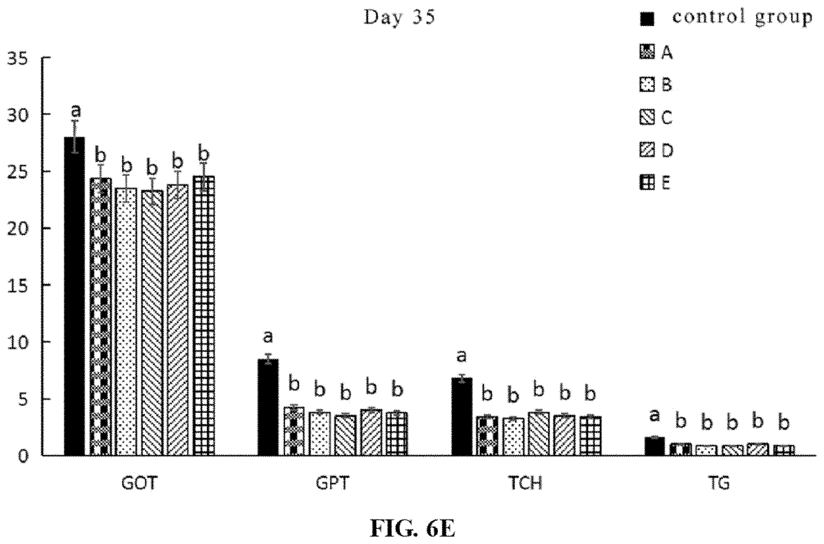

The serum biochemical index of tilapia were recorded on the 7th, 14th, 21st, 28th and 35th day after feeding with different formula feeds. The data of each group were compared by single factor analysis of variance. The results show that glutamic-oxaloacetate transaminase (GOT), glutamic-pyruvic transaminase (GPT), total cholesterol (TCH) and triglyceride (TG) of tilapia in the test groups are significantly lower than that in the control group, but there is no significant difference between the test groups (FIGS. 6A-6E). This indicates that different formula feeds has obvious effect of protecting liver.

4. Effects of Different Formula Feeds on Non-Specific Immune Index of Tilapia

Test method: the non-specific immune indexes of tilapia were determined according to the instructions of the relevant assay kit of Nanjing Jiancheng Bioengineering Institute. The determination included superoxide dismutase (SOD), malonaldehydic acid (MDA), activity of acid phosphatase (ACP), activity of lysozyme (LSY) and serum antibacterial activity.

3 tilapia were randomly selected from each group for collecting blood samples from the tail vein. The blood was centrifuged at 4000 r/min for 10 min after standing at 4° C. overnight. The supernatant was collected and stored at −80° C. for determining the activity of superoxide dismutase (SOD), peroxidase (POD), phenol oxidase (PO), lysozyme (LZM) and serum antibacterial activity.

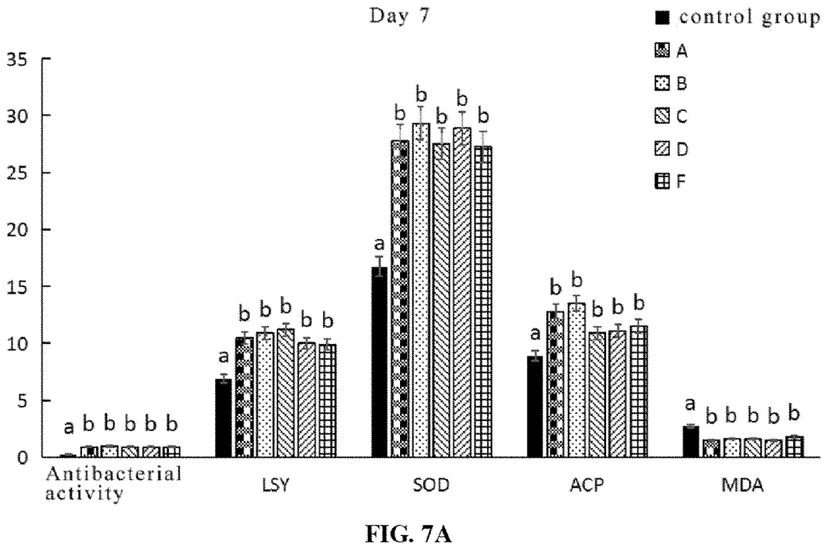

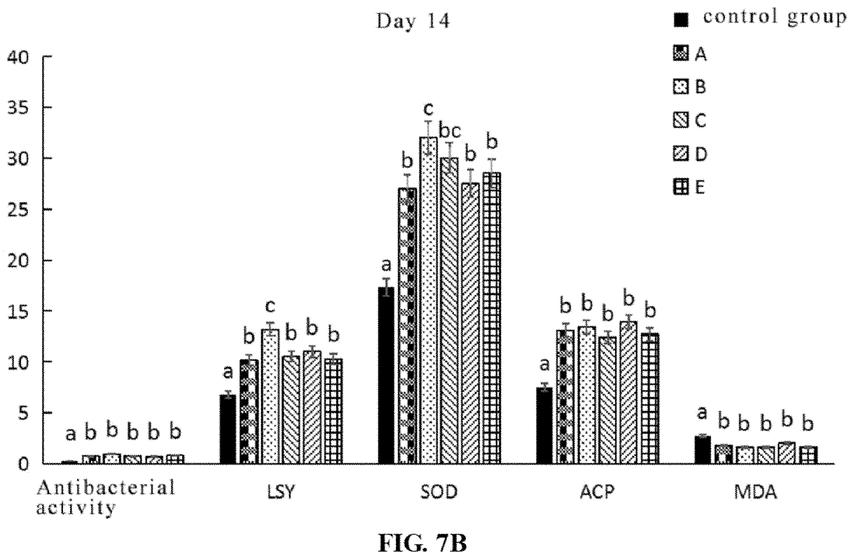

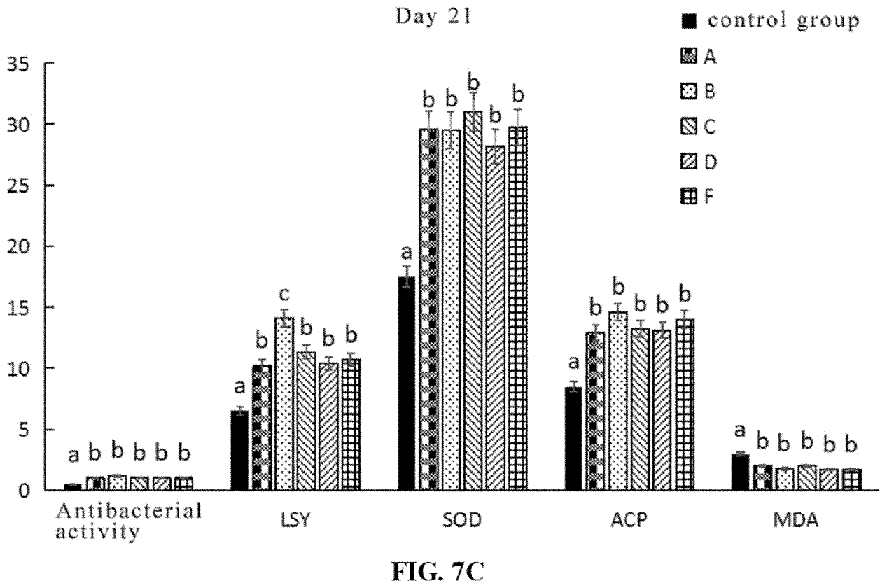

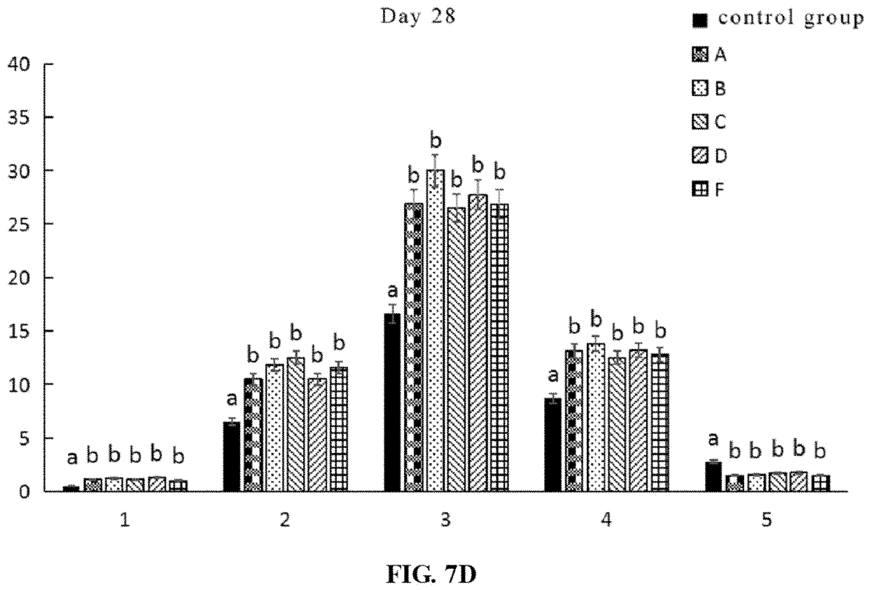

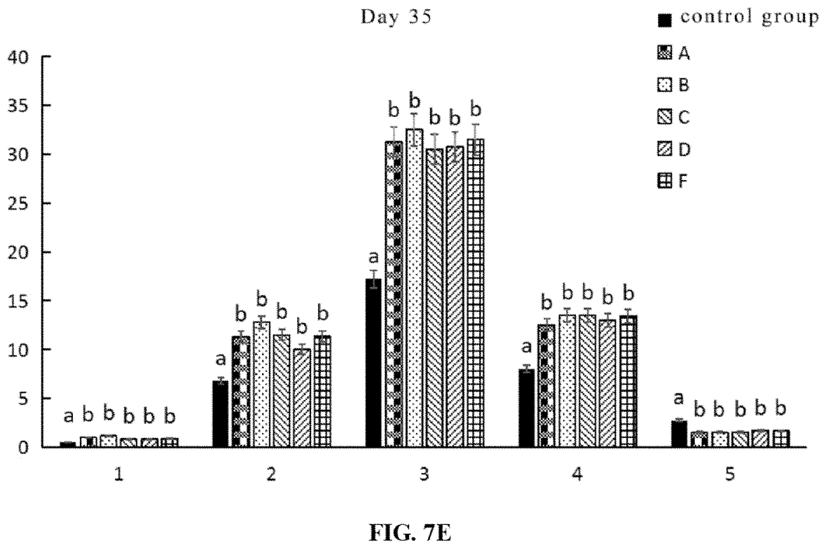

The serum immune index of tilapia was recorded on the 7th, 14th, 21st, 28th and 35th day after feeding with different formula feeds. The data of each group were compared by single factor analysis of variance. The results show that there are significant differences in serum antibacterial activity, LSY, SOD, ACP and MDA between the testing groups and control group (FIGS. 7A-7E). These results indicate that different formula feeds can enhance the non-specific immune function of tilapia and stress resistance; The activities of LSY and SOD in group B are significantly higher than those in other groups on the 14th and 21st day, indicating that the formula B might have better effect on improving the disease resistance of tilapia.

5. Effects of Different Formula Feeds on Intestinal Flora Index of Tilapia

Test method: 3 tilapia were randomly sampled from each group. The surface of tilapia was disinfected with 75% alcohol. The abdominal cavity was opened after disinfecting the clean bench once more. The internal abdominal cavity and external walls of intestine were wiped with alcohol and rinsed with sterile water for 3-5 times. The whole intestine was cut off with sterile scissors and rinsed with sterilized physiological saline for several times. The intestine was cut into pieces, then weighted to 1 g and transferred to the sterilized grinder under sterile conditions. The same amount of 0.85% sterilized physiological saline was added to grind the intestine into homogenate to make the stock solution. The stock solution was diluted by 10¹ to 10⁸ times in a sterilized centrifuge tube for later use. 100 μL diluents of different concentrations were inoculated on the medium by spread plate method. The total number of intestinal bacteria was counted by TTC medium, in particular, eosin-methylene blue medium (EMB) for Escherichia coli and Man Rogosa Sharpe medium (MRS) for Lactobacillus. Three replicates were made on each medium at each dilution, and the average was calculated.

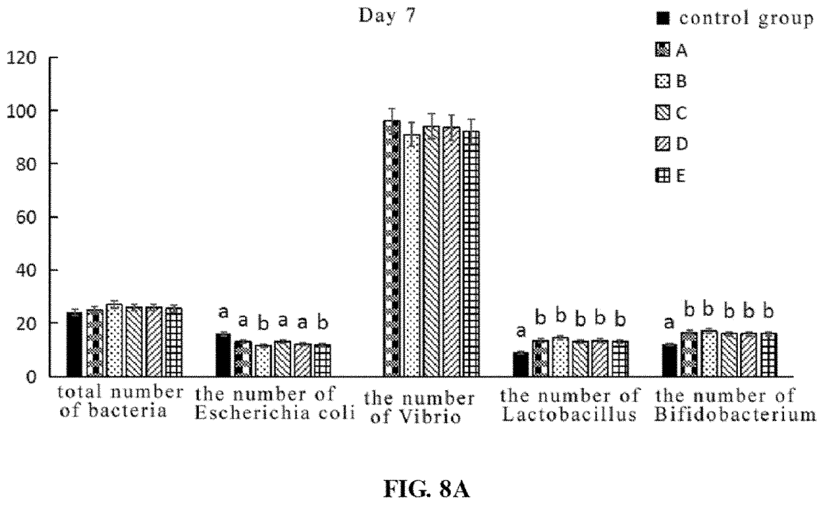

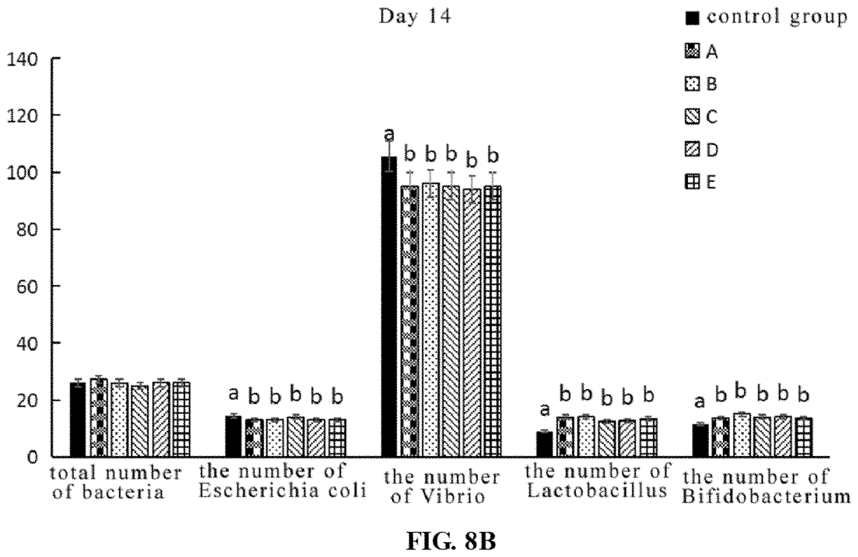

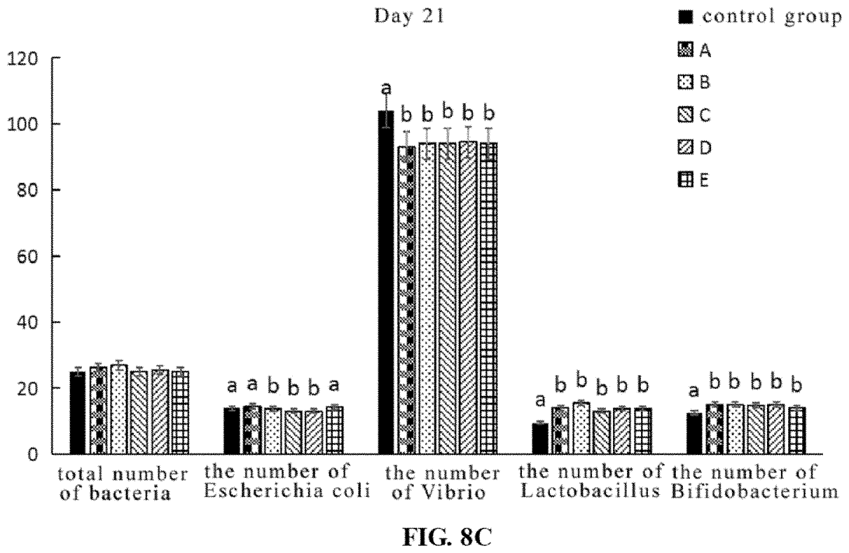

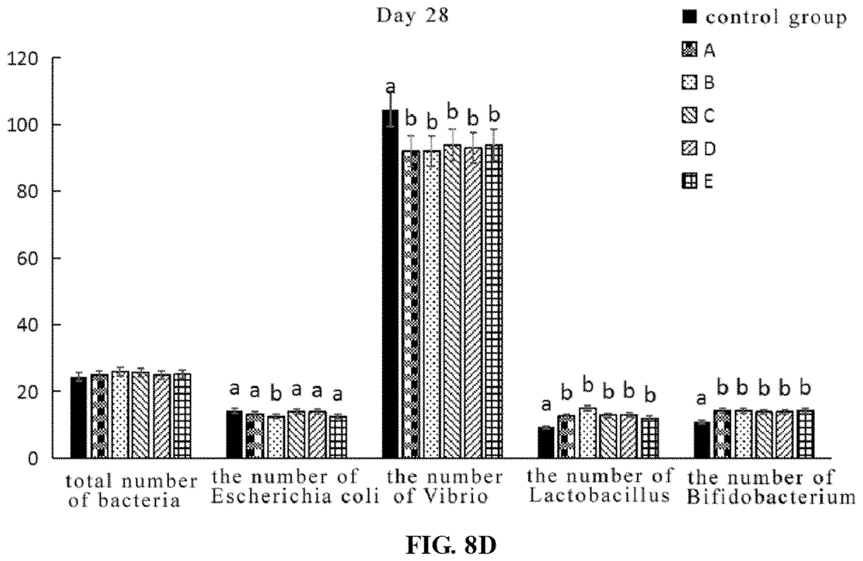

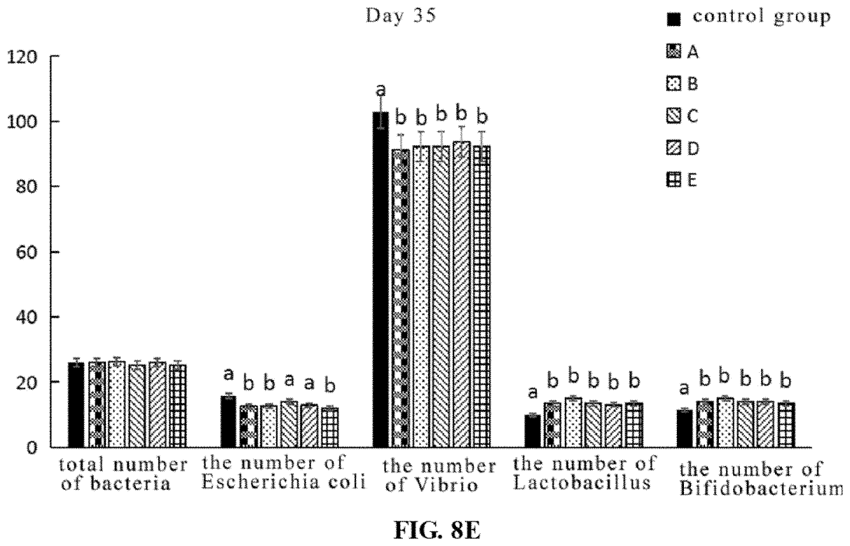

The intestinal flora index of tilapia were recorded on the 7th, 14th, 21st, 28th and 35th day after feeding with different formula feeds. The data of each group were compared by single factor analysis of variance. The results show that, compared with the control group, the number of Escherichia coli and Vibrio decrease significantly from the 14th day, while the number of probiotics Lactobacillus and Bifidobacterium increase significantly (FIGS. 8A-8E).

6. Effects of Different Formula Feeds on the Immune Protection of Tilapia Against Streptococcus agalactiae

Test method: tilapia was fed with different formula feeds. The tilapia was injected intramuscularly with 1×10⁸ CFU/mL Streptococcus agalactiae in the end of the test and cultured for 2 weeks. The mortality of tilapia was recorded, and the immune protection rate was calculated according to the formula of Amend (1981) (Amend DF. Potency testing of fish vaccines. In: International symposium on fish biologics: Serodiagnotics and vaccines. Dev Biol Stand, 1981, 49: 447-454), the immune protection was calculated according to the following Formula II:

RS=(1−mortality rate of immunization group %/mortality rate of control group %)×100 (Formula II)

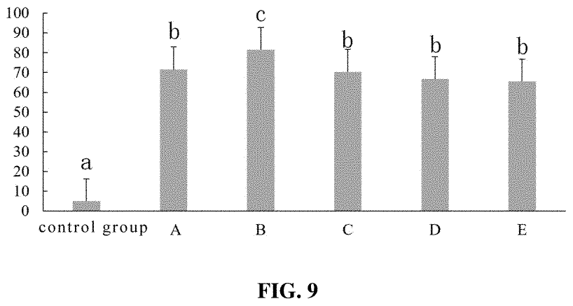

The results show that the highest immune protection rate in the control group is 93.82%, while the test groups A, B, C, D and E are 28.39%, 18.53%, 29.63%, 33.33% and 34.57%, respectively, which are significantly lower than those in the control group. The immune protection in the test groups are 71.63%, 81.48%, 70.37%, 66.67% and 65.63%, respectively, which are significantly different from control group, in particular, there is significant difference between group B and other test groups (P<0.05) (FIG. 9). In the challenge experiment, all the dead tilapia show the symptoms of infection with Streptococcus agalactiae, such as dermatorrhagia, abdominal swelling. The results indicate that the ability of anti Streptococcus agalactiae can be enhanced by feeding different formulas of compound Chinese herbal medicine, and the B formula of compound Chinese herbal medicine has the best effect.

According to the above results, the tilapia feed with formula B is the best feed for enhancing immunity, which can effectively increase the immune protection of tilapia against Streptococcus agalactiae.

The above is only preferred embodiments of the present disclosure, but the protection scope of the present disclosure is not limited to this. Any modifications, equivalent replacements and improvements made by anyone familiar with the technical field within the technical scope of the present disclosure and within the spirit and principle of the present disclosure shall be included in the protection scope of the present disclosure.

1. A feed for enhancing immunity of tilapia, wherein the feed comprises 20-30 parts of rice chaff, 15-25 parts of earthworm powder, 10-15 parts of shrimp shell powder, 10-15 parts of sodium alginate, 5-10 parts of oyster shell powder, 5-10 parts of cyclohexanone, 10-15 parts of honeysuckle extract, 3-5 parts of Commelina communis extract, 3-6 parts of β-1,3-glucan, 2-3 parts of flavomycin growth promoter, 1-3 parts of complex enzyme preparation, 5-8 parts of selenium methionine, 6-10 parts of distiller's grains, 1-3 parts of complex vitamins, 1-3 parts of complex trace elements and 2-3 parts of food attractant, in parts by weight.

2. The feed according to claim 1, wherein the complex enzyme preparation is a mixture of amylase, protease and cellulase, and the weight ratio of amylase, protease and cellulase is 3:2:1.

3. The feed according to claim 1, wherein the complex vitamins comprises two or more of vitamin C, vitamin E, vitamin K, vitamin A, vitamin D3, nicotinic acid, folic acid, inositol, and calcium pantothenate.

4. The feed according to claim 1, wherein the complex trace elements comprise iron, copper, manganese and iodine.

5. The feed according to claim 1, wherein the food attractant comprises allicin, seaweed, straw mushroom powder, dried silkworm pupa, clam meat, zeolite powder and fish soluble.

6. The feed according to claim 5, wherein a weight ratio of allicin, seaweed, straw mushroom powder, dried silkworm pupa, clam meat, zeolite powder and fish soluble is 2:2:1:1:1:1:2.

7. A method for preparing the feed for enhancing immunity of tilapia according to claim 1, wherein the method comprises the steps of:
S1: weighing rice chaff, earthworm powder, shrimp shell powder, sodium alginate, oyster shell powder, cyclohexanone, honeysuckle, Commelina communis, flavomycin growth promoter, complex enzyme preparation, selenium methionine, distiller's grains, complex vitamins and complex trace elements in proportion; preparing honeysuckle extract, Commelina communis extract and food attractant respectively;
S2: extracting β-1,3-glucan: mixing weighed rice chaff, earthworm powder, shrimp shell powder, and oyster shell powder evenly after removing impurities, crushing and passing through a 120-mesh sieve to obtain a mixed powder A;
S3: grinding sodium alginate, cyclohexanone and selenium methionine to obtain a mixture B; adding clear water, mixed powder A and mixture B to distiller's grains to obtain a mixture C after stirring evenly;
S4: adding flavomycin growth promoter, complex enzyme preparation, complex vitamins, complex trace elements to the mixture C to obtain feed for enhancing the immunity of tilapia after stirring evenly, granulating and drying.

8. The method according to claim 7, wherein a method for preparing the honeysuckle extract in S1 comprises the steps of:
(1) cleaning and drying the honeysuckle; cutting the dried honeysuckle into 1-3 cm segments, adding 15 times of water, and decocting the honeysuckle to obtain a primary decoction;
(2) vacuuming the primary decoction, heating to 60-80° C. to make the primary decoction boil, and keeping the temperature for 1-3 h, and then filtering to obtain a primary filtrate and a primary filter residue;
(3) adding extracting solvent to the primary filter residue for immersion and performing reflux extraction to obtain an extract; after filtering, mixing the filtered extract and the primary filtrate, concentrating the resulting mixture under reduced pressure, and drying the resulting concentrated solution to obtain a honeysuckle extract.

9. The method according to claim 8, wherein the ratio of the primary filter residue and extracting solvent in the step 3) is 1:15.

10. The method according to claim 8, wherein the parameters of reflux extraction in the step 3) are: temperature of the reflux extraction is 55° C.-95° C., extraction times are 2-3 times, and extraction time is 0.5 h-2 h.

11. The method according to claim 8, wherein the extracting solvent in the step 3) is an ethanol aqueous solution with a volume concentration of 75%.

12. The method according to claim 7, wherein a method for preparing the Commelina communis extract in S1 comprises the steps of:
firstly, steaming the Commelina communis at high temperature, collecting a steamed solution and a steamed Commelina communis;
secondly, putting the steamed Commelina communis into a beating machine to obtain a Commelina communis slurry;
then, heating and concentrating the steamed solution to obtain a concentrated solution;
finally, mixing the Commelina communis slurry and the concentrated solution to obtain a Commelina communis extract.

13. The method according to claim 12, wherein a temperature of steaming is 100° C.

14. The method according to claim 7, wherein a method for preparing the food attractant in S1 comprises the steps of:
1) weighing allicin, seaweed, straw mushroom powder, dried silkworm pupa, clam meat, zeolite powder and fish soluble; grinding allicin, seaweed, straw mushroom powder and zeolite powder, passing through a 140-mesh sieve to obtain a mixed powder;
2) injecting the fish soluble into a 500 L stainless steel vessel and steaming at high temperature for 30 min-60 min, natural cooling to 30° C.;
3) adding dried silkworm pupa and clam meat to a cooled fish soluble, mixing fully and adding trypsin and papain, mixing fully and fermenting at 37° C. for 3 h-5 h;
4) heating a fermentation product to boil for concentration until the water content is 63%, and cooling a concentrated fermentation product to 37° C.; adding mixed powder to a cooled mixture and drying in a vacuum drying oven to obtain a food attractant.

15. The method according to claim 7, wherein a method for extracting the β-1,3-glucan in the S2 comprises the steps of:
(2.1) washing, drying and crushing the mushroom, passing through a 120-mesh sieve to obtain a mushroom powder; dissolving the mushroom powder in 4-6 times volume of 100 mM phosphate buffer with pH 6.0-6.5, and extracting at 6° C. for 6 h;
(2.2) high-speed centrifuging to remove precipitate, saving supernatant to obtain a crude extract of β-1,3-glucan.
(2.3) adding a certain amount of NaOH into the crude extract of β-1,3-glucan, raising the temperature to 60° C.-75° C. to react for 2 h-3 h, collecting a precipitate by filtration;
(2.4) adding an acid solution to the precipitate, adjusting a pH to 5.5, raising a temperature to 65° C. to react for 1 h-2 h, collecting precipitate by filtration;
(2.5) washing the precipitate, extracting with acetone and freeze-drying to obtain a β-1,3-glucan.

16. The method according to claim 15, wherein a rotational speed of the high-speed centrifugation in the step (2.2) is 3000 rpm.

17. The method according to claim 15, wherein the amount of the NaOH in the step (2.3) added adjusts the pH of the crude extract to 6.0-6.5.

18. The method according to claim 7, wherein the complex enzyme preparation is a mixture of amylase, protease and cellulase, and the weight ratio of amylase, protease and cellulase is 3:2:1.

19. The method according to claim 7, wherein the complex vitamins comprises two or more of vitamin C, vitamin E, vitamin K, vitamin A, vitamin D3, nicotinic acid, folic acid, inositol, and calcium pantothenate.

20. The method according to claim 7, wherein the complex trace elements comprise iron, copper, manganese and iodine.
